(19) United States
(12) Patent Application Publication
(10) Pub. No.: US 2026/0315012 A1
(43) Pub. Date: Oct. 15, 2026

(54) INTEGRATED DETECTOR PLATFORM FOR X-RAY IMAGING

(71) Applicant: HealthTech Strategies, LLC, Philadelphia, PA (US)

(72) Inventor: Luca Bogoni, Philadelphia, PA (US)

(21) Appl. No.: 19/646,044

(22) Filed: Apr. 13, 2026

Related U.S. Application Data
(60) Provisional application No. 63/789,301, filed on Apr. 15, 2025.

(51) Int. Cl. A61B 6/04; A61B 6/10; A61B 6/42; A61B 6/58

(52) U.S. Cl. CPC A61B 6/0407; A61B 6/0487; A61B 6/107; A61B 6/4283; A61B 6/585

(57) ABSTRACT

An integrated detector platform embeds an X-ray detector within a structure having a bottom chassis and a radiolucent top surface defining a chamber therebetween. A displacement mechanism repositions the X-ray detector along one or more axes within the chamber. Supporting ribs between the chassis and top surface maintain structural integrity during detector movement, and a coupling mechanism maintains uniform rib spacing. A locking mechanism secures the detector at a selected position. A control system with motors and electronics drives the displacement mechanism based on positioning input and real-time feedback from sensors and encoders. Calibration features include a calibration grid in a slot aligned with a detector housing frame for coordinate transformation mapping and automatic detector identification with database-stored configurations. A scattering grid may be received in the same slot to reduce scatter radiation during imaging of larger patients. A safety interlock inhibits imaging when the detector is not locked.

### CROSS-REFERENCE TO RELATED APPLICATIONS

This application claims the benefit of U.S. Provisional Application No. 63/789,301, filed Apr. 15, 2025, the entire disclosure of which is incorporated herein by reference.

### FIELD

The present disclosure relates generally to medical imaging systems and, more particularly, to an X-ray detector platform integrated within a scanning surface for detector positioning and mechanical calibration.

### BACKGROUND

In conventional X-ray imaging rooms, patient-support tables feature floating designs that allow positioning of patients relative to fixed X-ray sources and detectors housed within Bucky assemblies beneath the table surface. In these configurations, either the table surface moves to align the patient with stationary imaging components, or the X-ray source and detector adjust positions to achieve a desired imaging alignment. Hybrid configurations combine both movable tables and adjustable imaging components.

While effective in controlled imaging suites, these configurations present challenges when applied to bedside imaging or scenarios involving immobile patients. In such cases, portable X-ray units are utilized and detectors are manually positioned beneath the patient. Manual placement can be labor-intensive and may pose risks of misalignment or inconsistent imaging results. Inadequate positioning can lead to pathological misinterpretation, unnecessary additional imaging procedures, patient anxiety, unnecessary pain, and increased radiation exposure.

Embedding an X-ray detector into a thin-profile patient-support surface offers a potential approach in which the system can maintain alignment with an X-ray source. Such an approach may be useful in environments such as intensive care units, patient wards, emergency departments, operating rooms, or home healthcare settings, where imaging capabilities are brought directly to the patient.

There remains an opportunity for a compact platform that can house and position an X-ray detector within a scanning table or bed structure while minimizing thickness, allowing precise positioning, and providing secure locking of the detector.

### SUMMARY

In accordance with one aspect, an integrated detector platform includes a bottom chassis, a radiolucent top surface disposed over the bottom chassis and defining a chamber therebetween, the radiolucent top surface forming a patient-support surface, an X-ray detector disposed within the chamber, a displacement mechanism operably coupled to the X-ray detector and configured to reposition the X-ray detector along at least one axis within the chamber, and a plurality of supporting ribs disposed within the chamber between the bottom chassis and the radiolucent top surface and configured to support the radiolucent top surface during displacement of the X-ray detector.

In some embodiments, the integrated detector platform further includes a locking mechanism configured to secure the X-ray detector at a selected position within the chamber. In some embodiments, the displacement mechanism includes at least one of a linear stage actuator, a rack and pinion mechanism, a belt and pulley mechanism, a lead screw mechanism, or a cam and follower mechanism. In some embodiments, the plurality of supporting ribs are associated with a coupling mechanism configured to maintain spacing among the supporting ribs during displacement of the X-ray detector. In some embodiments, the coupling mechanism includes at least one of a passive coupling using springs, an active coupling using motors, or a hybrid coupling combining passive and active elements. In some embodiments, the integrated detector platform further includes a control system operably connected to the displacement mechanism, the control system configured to receive a positioning input and to drive the displacement mechanism to position the X-ray detector based on the positioning input. In some embodiments, the bottom chassis incorporates shielding material configured to attenuate stray radiation. In some embodiments, the integrated detector platform further includes one or more sensors configured to provide real-time position feedback of the X-ray detector, enabling closed-loop control of the displacement mechanism. In some embodiments, the integrated detector platform further includes an access panel disposed on the integrated detector platform and configured to provide access to the chamber for insertion or removal of the X-ray detector. In some embodiments, the plurality of supporting ribs have a rectangular cross-section and are equipped with casters configured to facilitate movement of the supporting ribs during displacement of the X-ray detector. In some embodiments, the plurality of supporting ribs have a cylindrical cross-section with varying diameters configured to expand or contract with movement of the X-ray detector while preserving load distribution.

In accordance with another aspect, an integrated detector platform includes a bottom chassis comprising shielding material configured to attenuate stray radiation, a radiolucent top surface disposed over the bottom chassis and defining a chamber therebetween, the radiolucent top surface configured to permit transmission of X-rays therethrough and to support a patient, an X-ray detector disposed within the chamber, a displacement mechanism operably coupled to the X-ray detector and configured to reposition the X-ray detector along two orthogonal axes within the chamber, a plurality of supporting ribs disposed within the chamber between the bottom chassis and the radiolucent top surface, the plurality of supporting ribs configured to support the radiolucent top surface under load during displacement of the X-ray detector, a coupling mechanism associated with the plurality of supporting ribs and configured to maintain spacing among the supporting ribs as the X-ray detector displaces within the chamber, a locking mechanism configured to secure the X-ray detector at a selected position within the chamber, and a control system comprising motors and control electronics housed within the bottom chassis, the control system operably connected to the displacement mechanism and configured to drive the displacement mechanism based on positioning input.

In some embodiments, the integrated detector platform further includes sensors and encoders configured to provide real-time position feedback of the X-ray detector to the control system, enabling closed-loop control of the displacement mechanism. In some embodiments, the integrated detector platform further includes a calibration grid receivable in a slot aligned with a housing frame of the X-ray detector, the calibration grid configured for establishing a coordinate transformation between a coordinate system of the X-ray detector and a coordinate system of the integrated detector platform. In some embodiments, the coordinate transformation includes a nonlinear mapping accounting for positional discrepancies across an operational range of the X-ray detector. In some embodiments, the integrated detector platform is configured to automatically identify the X-ray detector upon connection and to retrieve pre-validated calibration configurations from a database associated with the identified X-ray detector. In some embodiments, the integrated detector platform further includes a safety interlock system configured to monitor a status of the locking mechanism and to inhibit imaging operations when the X-ray detector is not secured by the locking mechanism. In some embodiments, the displacement mechanism comprises a linear stage actuator.

In accordance with yet another aspect, an integrated detector platform includes a bottom chassis, a radiolucent top surface disposed over the bottom chassis and defining a chamber therebetween, the radiolucent top surface forming a patient-support surface, an X-ray detector disposed within the chamber, a displacement mechanism operably coupled to the X-ray detector and configured to reposition the X-ray detector along at least one axis within the chamber, a plurality of supporting ribs disposed within the chamber between the bottom chassis and the radiolucent top surface and configured to support the radiolucent top surface during displacement of the X-ray detector, a coupling mechanism associated with the plurality of supporting ribs and configured to maintain spacing among the supporting ribs during displacement of the X-ray detector, and a locking mechanism configured to secure the X-ray detector at a selected position within the chamber.

In some embodiments, the integrated detector platform further includes a control system operably connected to the displacement mechanism, the control system configured to receive a positioning input and to drive the displacement mechanism to position the X-ray detector based on the positioning input.

### BRIEF DESCRIPTION OF THE DRAWINGS

FIG. 1A is a top view and a lateral cross-section of an integrated detector platform according to an embodiment.

FIG. 1B is a block diagram of a control system housed within a bottom chassis of the integrated detector platform of FIG. 1A.

FIG. 2 is a detail perspective view and a lateral cross-section of an X-ray detector, supporting ribs, a displacement mechanism, and a calibration grid of the integrated detector platform of FIG. 1A.

FIG. 3A is a cross-sectional view of supporting ribs having a rectangular cross-section with casters and a coupling mechanism in a compressed state.

FIG. 3B is a cross-sectional view of the supporting ribs of FIG. 3A in an expanded state.

FIG. 3C is a cross-sectional view of supporting ribs having a cylindrical cross-section with a coupling mechanism in a compressed state.

FIG. 3D is a cross-sectional view of the supporting ribs of FIG. 3C in an expanded state.

FIG. 4A illustrates a supporting rib having a rectangular cross-section with uniform cylindrical segments.

FIG. 4B illustrates a supporting rib having a cylindrical cross-section with varying diameters.

FIG. 4C illustrates a supporting rib having disc-shaped stacked elements.

FIG. 5A is a schematic top view illustrating an X-ray detector in a first position within the integrated detector platform.

FIG. 5B is a schematic top view illustrating the X-ray detector in a second position displaced along a single axis.

FIG. 5C is a schematic top view illustrating the X-ray detector in a third position further displaced along the single axis.

FIG. 5D is a schematic top view illustrating the X-ray detector in a fourth position near a boundary of the integrated detector platform.

FIG. 6A is a schematic top view illustrating an X-ray detector in an initial position within the integrated detector platform prior to two-axis displacement.

FIG. 6B is a schematic top view illustrating the X-ray detector displaced along a first axis.

FIG. 6C is a schematic top view illustrating the X-ray detector displaced along a second axis orthogonal to the first axis.

### DETAILED DESCRIPTION OF EXAMPLE EMBODIMENTS

The following description sets forth example embodiments with reference to the drawings. The described embodiments are examples and are not intended to limit the scope of the claims. Features described in connection with one embodiment may be combined with features of other embodiments. Modifications and variations are possible without departing from the scope of the appended claims.

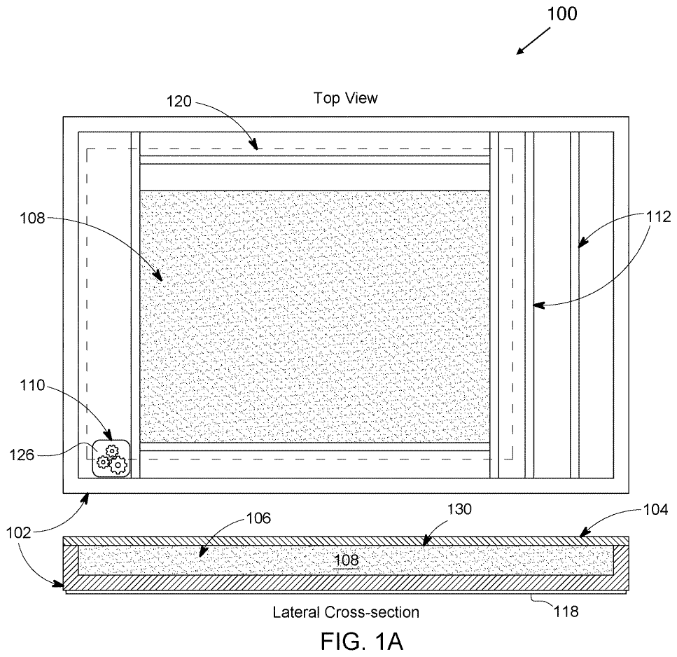

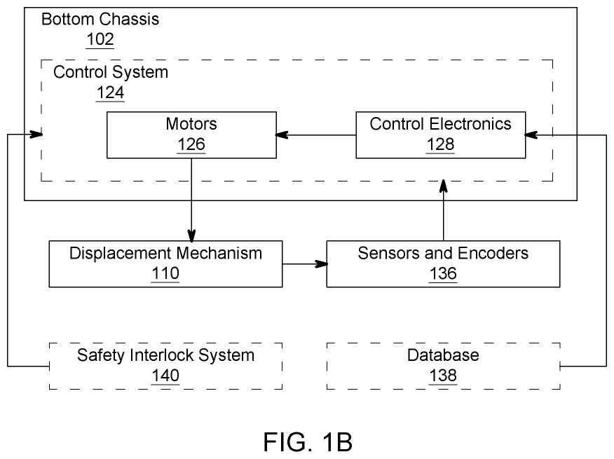

Referring to FIG. 1A, an integrated detector platform 100 is shown in a top view and a lateral cross-section. The integrated detector platform 100 provides a compact assembly for X-ray imaging that embeds the X-ray detector 108 within the structure rather than relying on a separate Bucky assembly beneath a table. The integrated detector platform 100 generally includes a bottom chassis 102, a radiolucent top surface 104 disposed over the bottom chassis 102 and defining a chamber 106 therebetween, an X-ray detector 108 disposed within the chamber 106, a displacement mechanism 110 operably coupled to the X-ray detector 108, and a plurality of supporting ribs 112 disposed within the chamber 106.

The bottom chassis 102 is configured for structural support of the integrated detector platform 100. The bottom chassis 102 is in the lateral cross-section of FIG. 1A as the U-shaped structural element forming the lower enclosure of the integrated detector platform 100. The bottom chassis 102 is typically manufactured from metals or rigid polymeric materials that do not require radiolucency. The bottom chassis 102 provides a rigid base to which internal components are mounted and serves as the structural foundation of the integrated detector platform 100. In some embodiments, the bottom chassis 102 incorporates shielding material 118 configured to attenuate stray radiation. In one embodiment, the shielding material 118 is integrated into the chassis material of the bottom chassis 102 during manufacturing. In another embodiment, the shielding material 118 is provided as a separate layer disposed within the bottom chassis 102 and bonded or laminated to an interior surface of the bottom chassis 102. The shielding material 118 includes thin lead or composite shielding to protect internal components and surrounding personnel from stray or backscattered X-rays. The shielding material 118 may comprise lead sheet, bismuth composite, or tungsten-impregnated polymer, selected based on thickness constraints and required attenuation levels. Exemplary thicknesses of the shielding material 118 range from approximately 0.5 mm to approximately 2.0 mm of lead equivalence, depending on the energy levels of the X-ray source and regulatory requirements for occupational exposure limits.

The radiolucent top surface 104 is disposed over the bottom chassis 102 and forms a patient-support surface. The radiolucent top surface 104 is configured to permit transmission of X-rays therethrough and to support a patient. The radiolucent top surface 104 is shown in the lateral cross-section of FIG. 1A as the upper panel of the integrated detector platform 100, positioned above the chamber 106. The radiolucent top surface 104 allows X-rays to pass through without significant attenuation. In various embodiments, the radiolucent top surface 104 is composed of a material selected from the group including carbon fiber composites, high-density polyethylene, thermoplastic composites reinforced with carbon fibers, and corrugated plastic structures. Carbon fiber composites provide strength and radiolucency. High-density polyethylene provides durability and resistance to staining, impact, and chemical corrosion while maintaining a smooth and easy-to-clean surface. Thermoplastic composites reinforced with carbon fibers combine the toughness of thermoplastics with the strength of carbon-fiber reinforcements, offering mechanical properties competitive with metals while maintaining radiolucency. Corrugated plastic structures, in which a flat plastic patient support sheet is backed by a slightly spaced corrugated sheet, can provide uniform electromagnetic wave attenuation and sufficient resistance to deflection.

The bottom chassis 102 and the radiolucent top surface 104 together define the chamber 106 therebetween. The chamber 106 is a hollow internal region that houses the X-ray detector 108, the displacement mechanism 110, the plurality of supporting ribs 112, and associated internal components. As shown in the lateral cross-section of FIG. 1A, the chamber 106 is bounded above by the radiolucent top surface 104 and below by the bottom chassis 102. The external components of the integrated detector platform 100, namely the bottom chassis 102 and the radiolucent top surface 104, collectively form a container enclosing the chamber 106.

The X-ray detector 108 is disposed within the chamber 106 between the bottom chassis 102 and the radiolucent top surface 104. As shown in FIG. 1A, the X-ray detector 108 is positioned in the top view within the X-ray detector 108 between the plurality of supporting ribs 112. The X-ray detector 108 is positioned to receive X-rays passing through the radiolucent top surface 104 and through a patient supported thereon. The X-ray detector 108 produces digital X-ray images that can be zoomed, manipulated, and transmitted for diagnostic evaluation. The X-ray detector 108 may be a flat panel detector or another suitable digital detector capable of generating diagnostic-quality images.

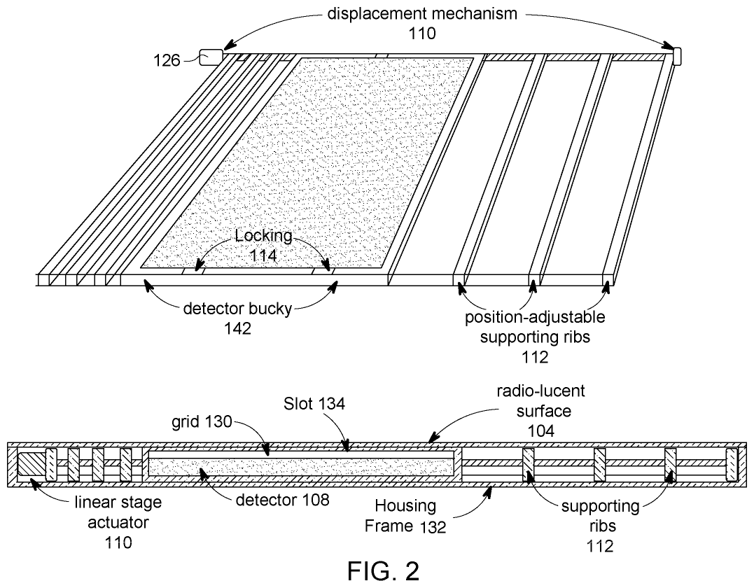

The displacement mechanism 110 is operably coupled to the X-ray detector 108 and configured to reposition the X-ray detector 108 along at least one axis within the chamber 106. As shown in FIG. 1A, the displacement mechanism 110 is located along a lower portion of the integrated detector platform 100 in the top view, represented by a gear icon. The displacement mechanism 110 is housed within the bottom chassis 102 and the radiolucent top surface 104, which have a limited separation in height. In an initial configuration, a default position of the X-ray detector 108 is in the middle of the range of travel. Various embodiments of the displacement mechanism 110 are described below in connection with FIG. 2.

The plurality of supporting ribs 112 are disposed within the chamber 106 between the bottom chassis 102 and the radiolucent top surface 104. As shown in FIG. 1A, the plurality of supporting ribs 112 appear as elongated elements extending across the integrated detector platform 100 above and below the X-ray detector 108 in the top view. The plurality of supporting ribs 112 are configured to support the radiolucent top surface 104 during displacement of the X-ray detector 108. The plurality of supporting ribs 112 maintain the structural integrity of the integrated detector platform 100 and preserve the space within the chamber 106 while sustaining load when a patient or weight is placed on the radiolucent top surface 104. The plurality of supporting ribs 112 allow the X-ray detector 108 to move within the chamber 106 without significant deformation of the radiolucent top surface 104.

Still referring to FIG. 1A, in some embodiments the integrated detector platform 100 further includes an access panel 120 disposed on the integrated detector platform 100 and configured to provide access to the chamber 106 for insertion, removal, or replacement of the X-ray detector 108. As shown in FIG. 1A, the access panel 120 is depicted as a dashed-outline region along the left side of the integrated detector platform 100 in the top view. In some embodiments, the access panel 120 includes a hinge mechanism on the radiolucent top surface 104 that allows the radiolucent top surface 104 to be opened as a lid. In other embodiments, the access panel 120 provides a drawer-like capability through which the X-ray detector 108 slides into and out of the chamber 106. The access panel 120 also grants maintenance access to internal actuators and cabling within the chamber 106.

Additional elements are visible in FIG. 1A. Motors 126 of the control system 124 are depicted within the bottom chassis 102 in the top view. The coupling mechanism 116 is depicted in the lateral cross-section between the plurality of supporting ribs 112 and the X-ray detector 108. The shielding material 118 is depicted in the lateral cross-section as a layer incorporated within the bottom chassis 102. The calibration grid 130 is depicted in the lateral cross-section received within the slot 134.

Turning to FIG. 1B, a block diagram illustrates the control system 124 housed within the bottom chassis 102 of the integrated detector platform 100. The control system 124 is operably connected to the displacement mechanism 110 and configured to receive a positioning input and to drive the displacement mechanism 110 to position the X-ray detector 108 based on the positioning input. As shown in FIG. 1B, the bottom chassis 102 is depicted as a solid-line outer container, and the control system 124 is depicted as a dashed-line inner container within the bottom chassis 102. The control system 124 comprises motors 126 and control electronics 128 housed within the bottom chassis 102.

The motors 126, shown as a block within the control system 124 in FIG. 1B, provide motive force to drive the displacement mechanism 110 and thereby reposition the X-ray detector 108 within the chamber 106. As illustrated by the connection arrow from the motors 126 to the displacement mechanism 110 in FIG. 1B, the motors 126 transmit mechanical force to the displacement mechanism 110 to effect controlled translation of the X-ray detector 108. In various embodiments, the motors 126 include stepper motors, servo motors, or brushless direct-current motors sized to produce sufficient torque within the height constraints of the chamber 106.

The control electronics 128, shown as a block adjacent to the motors 126 within the control system 124 in FIG. 1B, include controllers and associated circuitry that translate positioning input into control signals for the motors 126. As depicted by the connection arrow from the control electronics 128 to the motors 126 in FIG. 1B, the control electronics 128 generate command signals that drive the motors 126. The control system 124 utilizes standardized communication protocols including Ethernet/IP, RS-485, or wireless communication standards to facilitate integration with external imaging systems.

The control electronics 128 can include a processor and memory for storing instructions. Instructions for operation can be stored in the memory and executed by the processor to cause movement of the components of the platform 100 as disclosed herein. The platform 100 can be coupled to a cloud, server, or other device over a network and can be remotely controlled by the same.

Also shown in FIG. 1B are sensors and encoders 136 configured to provide real-time position feedback of the X-ray detector 108 to the control system 124, enabling closed-loop control of the displacement mechanism 110. The sensors and encoders 136 are depicted as an external block connected to the control system 124 by a feedback arrow in FIG. 1B. A connection arrow from the displacement mechanism 110 to the sensors and encoders 136 indicates that the sensors and encoders 136 monitor the position and movement of the displacement mechanism 110. The sensors and encoders 136 provide continuous feedback on the position and movement of the X-ray detector 108, and the real-time position feedback enables dynamic adjustments ensuring accurate alignment of the X-ray detector 108 with an X-ray source. The sensors and encoders 136 include one or more sensors coupled to the X-ray detector 108 or to the displacement mechanism 110, and the sensors and encoders 136 provide the real-time position feedback used for closed-loop control.

Referring now to FIG. 2, a detail perspective view and a lateral cross-section of the X-ray detector 108, the plurality of supporting ribs 112, and the displacement mechanism 110 within the integrated detector platform 100 are shown. The displacement mechanism 110 includes at least one of a linear stage actuator, a rack and pinion mechanism, a belt and pulley mechanism, a lead screw mechanism, or a cam and follower mechanism. As depicted in FIG. 2, the displacement mechanism 110 is shown as a linear stage actuator positioned at opposite edges of the X-ray detector 108, labeled at both the left-side perspective view and the lower portion of the lateral cross-section. Linear stage actuators can precisely control movement in the X and Y directions and offer high precision and repeatability for applications requiring exact positioning of the X-ray detector 108. A rack and pinion mechanism converts rotational motion into linear displacement and provides a robust and cost-effective solution. A belt and pulley mechanism provides smooth and quick displacement and allows for high-speed movement of the X-ray detector 108. A lead screw mechanism driven by a motor can precisely position the X-ray detector 108 along a specified axis with high accuracy and repeatability. A cam and follower mechanism translates rotational motion into linear displacement and provides controlled incremental movement of the X-ray detector 108. Each of these mechanisms offers different advantages in terms of precision, cost, complexity, and speed of movement, and the choice of mechanism can be tailored to specific application requirements.

As further shown in FIG. 2, a detector bucky 142 is a carriage assembly disposed within the chamber 106 and configured to receive and retain the X-ray detector 108. The displacement mechanism 110 is operably coupled to the detector bucky 142 such that the displacement mechanism 110 repositions the detector bucky 142 and the X-ray detector 108 together within the chamber 106. The housing frame 132 is a structural component of the detector bucky 142 surrounding the X-ray detector 108, and the slot 134 is aligned with the housing frame 132 of the detector bucky 142.

Continuing with FIG. 2, a locking mechanism 114 is configured to secure the X-ray detector 108 at a selected position within the chamber 106. As labeled in FIG. 2, the locking mechanism 114 is shown in the perspective view at a lower portion of the X-ray detector 108. The locking mechanism 114 ensures stability of the X-ray detector 108 during image acquisition. In various embodiments, the locking mechanism 114 includes at least one of a mechanical latch, an electromagnetic lock, or a spring-loaded clamp. A mechanical latch can be manually or motor-driven and engages a catch on a housing of the X-ray detector 108. An electromagnetic lock is activated and deactivated via electrical current to lock the X-ray detector 108 in place, allowing repositioning of the X-ray detector 108 when the electromagnetic lock is released. A spring-loaded clamp automatically engages under a neutral or powered-off state for safety and employs spring tension to hold the X-ray detector 108 firmly. The locking mechanism 114 is configured to be engaged and disengaged to allow quick replacement or repositioning of the X-ray detector 108 while maintaining safety and stability during imaging use.

Also in the lateral cross-section of FIG. 2 is a calibration grid 130. The calibration grid 130 is receivable in a slot 134 aligned with the housing frame 132 of the detector bucky 142 surrounding the X-ray detector 108. The slot 134 is shown in the lateral cross-section of FIG. 2 between the radiolucent top surface 104 and the X-ray detector 108. The housing frame 132 is shown in the lateral cross-section of FIG. 2 as a structural component surrounding the X-ray detector 108. The calibration grid 130 is configured for establishing a coordinate transformation between a coordinate system of the X-ray detector 108 and a coordinate system of the integrated detector platform 100. During the calibration phase, the X-ray detector 108 is moved to predefined reference positions within the integrated detector platform 100, and the calibration grid 130 is used to generate a spatial mapping so that images can be corrected for positional discrepancies. Fiducial points such as corners of the X-ray detector 108 are aligned with the calibration grid 130, and drifts from expected positions are accounted for in conjunction with position data from the sensors and encoders 136. In some embodiments, the calibration grid 130 is present in the integrated detector platform 100 during the calibration phase and is removed before imaging operations, as the calibration grid 130 may leave imaging traces if left in place during exposure. In one embodiment, the slot 134 is an internal slot designed for the calibration grid 130, ensuring precise alignment with the housing frame 132 of the X-ray detector 108. In another embodiment, an external clamp or fixture can secure the calibration grid 130 on a surface of the integrated detector platform 100, aligning the calibration grid 130 with a known coordinate reference system. The housing frame 132 provides a mechanical reference point for alignment of the calibration grid 130 and ensures that the position of the calibration grid 130 is fixed relative to the X-ray detector 108, eliminating dependencies on external X-ray system configurations. The calibration process establishes the coordinate transformation that aligns the coordinate system of the X-ray detector 108 with the coordinate system of the integrated detector platform 100. While a simple linear transformation involving translations and rotation may be used, in some embodiments the coordinate transformation includes a nonlinear mapping accounting for positional discrepancies across an operational range of the X-ray detector 108. The nonlinear mapping addresses variations in mechanical response across the full span of travel of the X-ray detector 108.

Also visible in FIG. 2 are motors 126 of the control system 124 disposed within the bottom chassis 102 adjacent to the displacement mechanism 110, and the radiolucent top surface 104 above the X-ray detector 108.

In some embodiments, the slot 134 is configured to receive an insertable grid structure, the insertable grid structure comprising at least one of the calibration grid 130 or a scattering grid. In such embodiments, the slot 134 receives the scattering grid in place of the calibration grid 130 during imaging operations. The scattering grid is an optional component receivable in the same slot 134 aligned with the housing frame 132 of the X-ray detector 108. The scattering grid is configured to reduce scatter radiation during imaging, particularly when the integrated detector platform 100 is used to position the X-ray detector 108 beneath patients exceeding approximately 200 pounds and up to approximately 600 pounds. In such cases, the body mass of the patient increases the amount of scattered radiation reaching the X-ray detector 108, and the scattering grid guides incident X-rays toward the X-ray detector 108 while attenuating off-axis scattered photons. The scattering grid improves image contrast and diagnostic quality in these higher-mass patient scenarios. In some embodiments, virtual scattering correction using artificial intelligence algorithms may be employed as an alternative to or in combination with a physical scattering grid, reducing the need for a physical scattering grid by computationally correcting for scatter artifacts. The calibration grid 130 and the scattering grid serve functionally distinct roles: the calibration grid 130 is used during a setup phase to establish coordinate mapping and is then removed, while the scattering grid is used during imaging operations to reduce scatter and remains in the slot 134 throughout the imaging procedure.

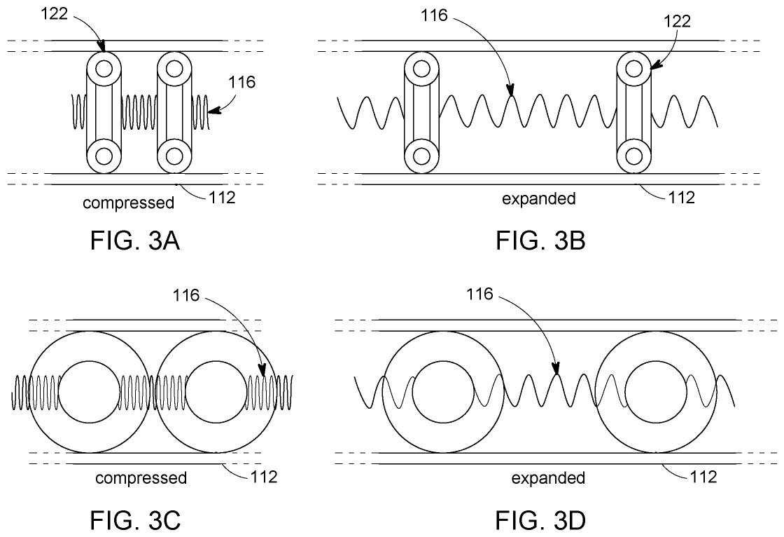

As further shown in FIG. 2, the plurality of supporting ribs 112 are labeled in the perspective view as position-adjustable elements extending across the width of the integrated detector platform 100 on both sides of the X-ray detector 108, and in the lateral cross-section as elements disposed between the radiolucent top surface 104 and the bottom chassis 102. The plurality of supporting ribs 112 contact the underside of the radiolucent top surface 104 and rest upon the bottom chassis 102, spanning the height of the chamber 106. Each of the plurality of supporting ribs 112 is free to translate laterally within the chamber 106 as the X-ray detector 108 is repositioned by the displacement mechanism 110, as described below in connection with FIGS. 3A through 6C.

Referring now to FIGS. 3A through 3D, cross-sectional views illustrate a coupling mechanism 116 for the plurality of supporting ribs 112 in compressed and expanded states. The coupling mechanism 116 is associated with the plurality of supporting ribs 112 and configured to maintain spacing among the supporting ribs 112 during displacement of the X-ray detector 108.

In FIG. 3A, the plurality of supporting ribs 112 have a rectangular cross-section and are equipped with casters 122 configured to facilitate movement of the supporting ribs 112 during displacement of the X-ray detector 108. As shown in FIG. 3A, the coupling mechanism 116 comprises springs disposed between adjacent supporting ribs 112, maintaining even spacing among the plurality of supporting ribs 112 as the X-ray detector 108 moves within the chamber 106. FIG. 3A depicts a compressed state in which the plurality of supporting ribs 112 are spaced closer together. The casters 122, shown at the lower edge of each rectangular supporting rib 112 in FIG. 3A, allow each of the supporting ribs 112 to slide or roll as spacing changes due to displacement of the X-ray detector 108.

In FIG. 3B, the same rectangular supporting ribs 112 with casters 122 are shown in an expanded state in which the plurality of supporting ribs 112 are spaced further apart. The coupling mechanism 116 sustains uniform mechanical support across the radiolucent top surface 104 in both the compressed state of FIG. 3A and the expanded state of FIG. 3B. When the X-ray detector 108 shifts in a positive direction, the supporting ribs 112 on one side compress while the supporting ribs 112 on the opposite side expand, and the coupling mechanism 116 sustains uniform mechanical support across the radiolucent top surface 104. The casters 122 provide a smooth sliding mechanism that ensures stability and ease of movement.

In FIG. 3C, the plurality of supporting ribs 112 have a cylindrical cross-section with varying diameters configured to expand or contract with movement of the X-ray detector 108 while preserving load distribution. As shown in FIG. 3C, the coupling mechanism 116 again comprises springs disposed between adjacent cylindrical supporting ribs 112, serving the same coupling function as in FIGS. 3A and 3B and maintaining uniform spacing. FIG. 3C depicts a compressed state in which the cylindrical supporting ribs 112 are spaced closer together.

In FIG. 3D, the same cylindrical supporting ribs 112 are shown in an expanded state. The varying diameters allow a compressible structure that adjusts with movement of the X-ray detector 108 while preserving load distribution. The cylindrical cross-section offers flexibility in coupling, allowing adaptability to different patient weights and surface conditions.

In some embodiments, the coupling mechanism 116 includes a passive coupling using springs as shown in FIGS. 3A through 3D. In some embodiments, the coupling mechanism 116 includes an active coupling using motors that dynamically adjust the position or orientation of the plurality of supporting ribs 112 in synchronization with movement of the X-ray detector 108. In some embodiments, the coupling mechanism 116 includes a hybrid coupling combining passive and active elements to balance responsiveness and control, adapting to different operational requirements.

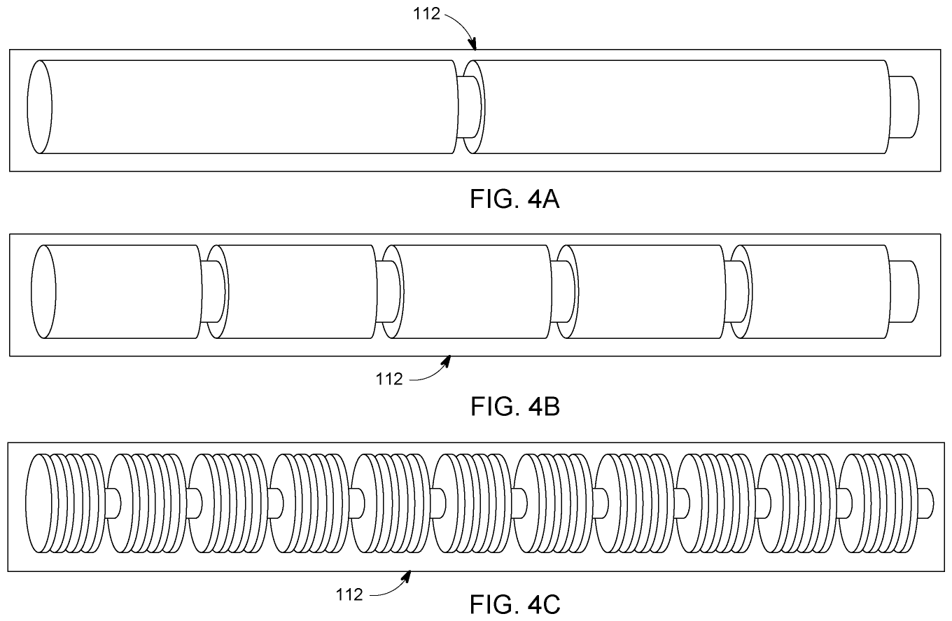

As illustrated in FIGS. 4A through 4C, the plurality of supporting ribs 112 can be configured in various cross-sectional profiles. In FIG. 4A, the plurality of supporting ribs 112 have a rectangular cross-section with uniform cylindrical segments. As shown in FIG. 4A, each of the supporting ribs 112 comprises two cylindrical segments of equal diameter joined at a junction, producing a rib with a uniform profile along its length.

In FIG. 4B, the plurality of supporting ribs 112 have a cylindrical cross-section with varying diameters. As shown in FIG. 4B, each of the supporting ribs 112 comprises multiple cylindrical segments of progressively smaller diameter stacked vertically, producing a tapered or telescoping profile that allows controlled compression.

In FIG. 4C, the plurality of supporting ribs 112 include disc-shaped or bellows-style stacked elements that provide compressibility along the length of the rib. As shown in FIG. 4C, each of the supporting ribs 112 comprises a series of thin disc elements closely spaced along the vertical axis, producing a structure that can compress and expand in an accordion-like manner.

The choice of rib design depends on specific application requirements, balancing factors such as manufacturing complexity, cost, stability, and weight-bearing capacity. In some embodiments, the plurality of supporting ribs 112 include small wheel arrays that minimize friction during movement of the X-ray detector 108. In some configurations, wheel sizing and distribution are selected to balance friction reduction and load-bearing capacity.

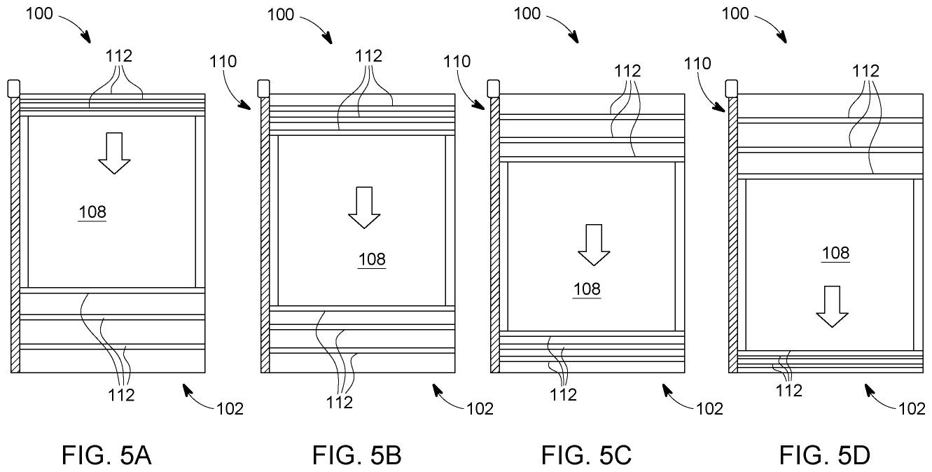

Referring to FIGS. 5A through 5D, a series of schematic top views illustrate progressive displacement of the X-ray detector 108 along a single axis within the integrated detector platform 100. The four positions shown in FIGS. 5A through 5D are representative of a continuous range of travel; the displacement mechanism 110 can position the X-ray detector 108 at any point along the axis, not only at the four discrete positions depicted. As the displacement mechanism 110 drives the X-ray detector 108 along the axis, the plurality of supporting ribs 112 passively redistribute within the chamber 106, compressing on the side toward which the X-ray detector 108 advances and expanding on the opposite side, while the coupling mechanism 116 maintains uniform spacing among the plurality of supporting ribs 112 throughout the travel.

FIGS. 5A through 5D illustrate the integrated detector platform 100 with the X-ray detector 108 positioned at four sequential positions along a single axis. The bottom chassis 102 is visible in each of FIGS. 5A through 5D as the structural enclosure of the integrated detector platform 100. Although not separately labeled in FIGS. 5A through 5D, the coupling mechanism 116 is shown in FIGS. 3A through 3D.

In FIG. 5A, the X-ray detector 108 is in a first position within the integrated detector platform 100. The plurality of supporting ribs 112 are distributed on both sides of the X-ray detector 108. A directional arrow within the X-ray detector 108 indicates the direction of impending movement. The coupling mechanism 116 is between the plurality of supporting ribs 112 and the X-ray detector 108.

In FIG. 5B, the X-ray detector 108 has moved to a second position further along the axis in the direction indicated by the arrow. The plurality of supporting ribs 112 to the left of the X-ray detector 108 have compressed, as evidenced by the reduced spacing, and the coupling mechanism 116 is between the X-ray detector 108 and the compressed supporting ribs 112.

In FIG. 5C, the X-ray detector 108 has moved to a third position still further along the axis. The plurality of supporting ribs 112 to the left of the X-ray detector 108 are further compressed, and the plurality of supporting ribs 112 to the right of the X-ray detector 108 have expanded to occupy the vacated space.

In FIG. 5D, the X-ray detector 108 has moved to a fourth position near a boundary of the integrated detector platform 100. The plurality of supporting ribs 112 to the left of the X-ray detector 108 are at maximum compression, while the plurality of supporting ribs 112 to the right of the X-ray detector 108 occupy a minimal region. The coupling mechanism 116 maintains separation among the plurality of supporting ribs 112 to apply even support to the radiolucent top surface 104 across the full range of travel of the X-ray detector 108. The displacement mechanism 110 provides controlled movement with accuracy and relatively real-time smooth position control within the overall space constraints of the chamber 106.

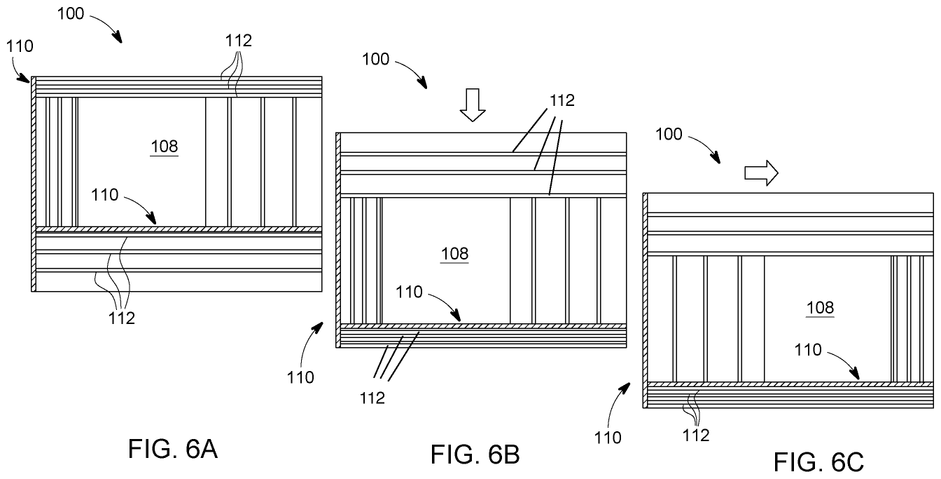

As shown in FIG. 6A, the X-ray detector 108 is in an initial position within the integrated detector platform 100, with the plurality of supporting ribs 112 distributed on all sides of the X-ray detector 108 and the coupling mechanism 116 (see FIGS. 3A through 3D) between the X-ray detector 108 and the plurality of supporting ribs 112. Two displacement mechanisms 110 are configured to reposition the X-ray detector 108 along two orthogonal axes within the integrated detector platform 100.

In FIG. 6B, the X-ray detector 108 has displaced along a first axis, and the plurality of supporting ribs 112 have adjusted spacing accordingly. In FIG. 6C, the X-ray detector 108 has displaced along a second axis orthogonal to the first axis, and the plurality of supporting ribs 112 have further adjusted. The plurality of supporting ribs 112 can displace in both horizontal and vertical directions in response to movement of the X-ray detector 108. In practice, displacement of the plurality of supporting ribs 112 may be implemented incrementally in both directions. In some embodiments, a single coupling mechanism 116 (see FIGS. 3A through 3D) coordinates the spacing of the plurality of supporting ribs 112 along both orthogonal axes, managing rib spacing in the space between the X-ray detector 108 and the internal walls of the integrated detector platform 100 regardless of the direction of displacement. In other embodiments, multiple coupling mechanisms 116 are provided, with a first coupling mechanism 116 coordinating spacing of the plurality of supporting ribs 112 along the first axis and a second coupling mechanism 116 coordinating spacing of the plurality of supporting ribs 112 along the second axis, such that each coupling mechanism 116 independently manages rib distribution in its respective direction of displacement. In either configuration, the coupling mechanism 116 ensures that the radiolucent top surface 104 remains uniformly supported under load during displacement of the X-ray detector 108 along both axes. Again, two displacement mechanisms 110 are configured to reposition the X-ray detector 108 along two orthogonal axes within the integrated detector platform 100.

Referring again to FIG. 1B, during calibration the control system 124 moves the X-ray detector 108 to predefined reference positions within the integrated detector platform 100 using two displacement mechanisms 110. At each position, the control system 124 verifies that the actual location of the X-ray detector 108 matches intended coordinates, ensuring that the two displacement mechanisms 110 provides accurate and repeatable movements. The control electronics 128 compare commanded positions with position data received from the sensors and encoders 136 and issue corrective commands to the motors 126 as needed. Performance verification after calibration includes test scans to validate performance of the X-ray detector 108, checking for artifacts, ensuring consistent image quality, and verifying that calibration parameters are correctly applied.

In some embodiments, the integrated detector platform 100 is configured to automatically identify the X-ray detector 108 upon connection and to retrieve pre-validated calibration configurations from a database 138 associated with the identified X-ray detector 108. The database 138 is shown as a dashed-line block in FIG. 1B connected to the control system 124. The database 138 stores configuration information including calibration configurations for multiple detectors for which the integrated detector platform 100 has been previously tested and for which factory calibration has been performed. The integrated detector platform 100 identifies the X-ray detector 108 by reading a serial number or unique identifier from the X-ray detector 108 via an electrical or communication interface upon connection, and the control electronics 128 query the database 138 using the identifier to retrieve the corresponding pre-validated calibration configurations. In some embodiments, the database 138 is stored locally on the control electronics 128. In other embodiments, the database 138 is hosted on a networked resource accessible via the communication protocols of the control system 124. A web-based user interface is available to allow users to manually input or adjust details of the X-ray detector 108, providing flexibility in managing configurations and calibration settings for the X-ray detector 108.

In some embodiments, the integrated detector platform 100 further includes a safety interlock system 140 configured to monitor a status of the locking mechanism 114 and to inhibit imaging operations when the X-ray detector 108 is not secured by the locking mechanism 114. The safety interlock system 140 is shown as a dashed-line block in FIG. 1B connected to the control system 124. The safety interlock system 140 provides a fail-safe function that prevents imaging from proceeding under conditions in which the X-ray detector 108 might move during exposure. The safety interlock system 140 may include redundant power supplies and emergency stop functions to maintain system integrity during unexpected events. The safety interlock system 140 communicates with the control system 124 such that the control electronics 128 prevent the motors 126 from driving the displacement mechanism 110 when the locking mechanism 114 is in a locked state during an imaging operation.

The integrated detector platform 100 supports preventive maintenance including drift detection, movement verification, and periodic coordinate transformation assessment. At scheduled intervals based on usage intensity, the control system 124 performs functional verification of the displacement mechanism 110, confirming that the X-ray detector 108 moves accurately to designated positions without unexpected delays or resistance. Calibration verification uses calibrated phantoms to assess image quality and alignment, and the control system 124 compares current calibration data with baseline measurements to identify shifts in the coordinate transformation, including both linear and nonlinear drifts. Mechanical inspection of components such as gears, bearings, and actuators addresses wear, corrosion, or misalignment. The sensors and encoders 136 are tested for accuracy against known standards, and methods such as discrete wavelet transform and grey models may be employed for drift detection in the sensors and encoders 136. Software and firmware updates are applied to incorporate performance improvements. Maintenance records are maintained for tracking performance of the integrated detector platform 100 over time.

The integrated detector platform 100 is configured for use in various clinical settings, including intensive care units, emergency departments, operating rooms, and long-term care facilities, to facilitate in-situ imaging without necessitating patient transport. The integrated detector platform 100 reduces the overall thickness of the assembly and enables easier under-patient placement with less disturbance. The ability to displace the X-ray detector 108 within the chamber 106 using the displacement mechanism 110, lock the X-ray detector 108 via the locking mechanism 114, and calibrate via the calibration grid 130 provides a workflow that minimizes patient repositioning and reduces the likelihood of misalignment.

While embodiments have been described with reference to specific configurations, the scope of the disclosure is not limited to these configurations. Various modifications and variations are possible without departing from the scope of the appended claims. Accordingly, the specification and drawings are to be regarded in an illustrative rather than a restrictive sense, and all modifications are intended to fall within the scope of the appended claims.

What is claimed is:

1. An integrated detector platform comprising:
a bottom chassis;
a radiolucent top surface disposed over the bottom chassis and defining a chamber therebetween, the radiolucent top surface forming a patient-support surface;
an X-ray detector disposed within the chamber;
a displacement mechanism operably coupled to the X-ray detector and configured to reposition the X-ray detector along at least one axis within the chamber; and
a plurality of supporting ribs disposed within the chamber between the bottom chassis and the radiolucent top surface and configured to support the radiolucent top surface during displacement of the X-ray detector.

2. The integrated detector platform of claim 1, further comprising a locking mechanism configured to secure the X-ray detector at a selected position within the chamber.

3. The integrated detector platform of claim 1, wherein the displacement mechanism comprises at least one of a linear stage actuator, a rack and pinion mechanism, a belt and pulley mechanism, a lead screw mechanism, or a cam and follower mechanism.

4. The integrated detector platform of claim 1, wherein the plurality of supporting ribs are associated with a coupling mechanism configured to maintain spacing among the supporting ribs during displacement of the X-ray detector.

5. The integrated detector platform of claim 4, wherein the coupling mechanism comprises at least one of a passive coupling using springs, an active coupling using motors, or a hybrid coupling combining passive and active elements.

6. The integrated detector platform of claim 1, further comprising a control system operably connected to the displacement mechanism, the control system configured to receive a positioning input and to drive the displacement mechanism to position the X-ray detector based on the positioning input.

7. The integrated detector platform of claim 1, wherein the bottom chassis incorporates shielding material configured to attenuate stray radiation.

8. The integrated detector platform of claim 1, further comprising one or more sensors configured to provide real-time position feedback of the X-ray detector, enabling closed-loop control of the displacement mechanism.

9. The integrated detector platform of claim 1, further comprising an access panel disposed on the integrated detector platform and configured to provide access to the chamber for insertion or removal of the X-ray detector.

10. The integrated detector platform of claim 1, wherein the plurality of supporting ribs have a rectangular cross-section and are equipped with casters configured to facilitate movement of the supporting ribs during displacement of the X-ray detector.

11. The integrated detector platform of claim 1, wherein the plurality of supporting ribs have a cylindrical cross-section with varying diameters configured to expand or contract with movement of the X-ray detector while preserving load distribution.

12. An integrated detector platform comprising:
a bottom chassis comprising shielding material configured to attenuate stray radiation;
a radiolucent top surface disposed over the bottom chassis and defining a chamber therebetween, the radiolucent top surface configured to permit transmission of X-rays therethrough and to support a patient;
an X-ray detector disposed within the chamber;
a displacement mechanism operably coupled to the X-ray detector and configured to reposition the X-ray detector along two orthogonal axes within the chamber;
a plurality of supporting ribs disposed within the chamber between the bottom chassis and the radiolucent top surface, the plurality of supporting ribs configured to support the radiolucent top surface under load during displacement of the X-ray detector;
a coupling mechanism associated with the plurality of supporting ribs and configured to maintain spacing among the supporting ribs as the X-ray detector displaces within the chamber;
a locking mechanism configured to secure the X-ray detector at a selected position within the chamber; and
a control system comprising motors and control electronics housed within the bottom chassis, the control system operably connected to the displacement mechanism and configured to drive the displacement mechanism based on positioning input.

13. The integrated detector platform of claim 12, further comprising sensors and encoders configured to provide real-time position feedback of the X-ray detector to the control system, enabling closed-loop control of the displacement mechanism.

14. The integrated detector platform of claim 12, further comprising a slot aligned with a housing frame of the X-ray detector and configured to receive an insertable grid structure, the insertable grid structure comprising at least one of a calibration grid configured for establishing a coordinate transformation between a coordinate system of the X-ray detector and a coordinate system of the integrated detector platform, or a scattering grid configured to reduce scatter radiation during imaging.

15. The integrated detector platform of claim 14, wherein the coordinate transformation comprises a nonlinear mapping accounting for positional discrepancies across an operational range of the X-ray detector.

16. The integrated detector platform of claim 12, wherein the integrated detector platform is configured to automatically identify the X-ray detector upon connection and to retrieve pre-validated calibration configurations from a database associated with the identified X-ray detector.

17. The integrated detector platform of claim 12, further comprising a safety interlock system configured to monitor a status of the locking mechanism and to inhibit imaging operations when the X-ray detector is not secured by the locking mechanism.

18. The integrated detector platform of claim 12, wherein the displacement mechanism comprises a linear stage actuator.

19. An integrated detector platform comprising:
a bottom chassis;
a radiolucent top surface disposed over the bottom chassis and defining a chamber therebetween, the radiolucent top surface forming a patient-support surface;
an X-ray detector disposed within the chamber;
a displacement mechanism operably coupled to the X-ray detector and configured to reposition the X-ray detector along at least one axis within the chamber;
a plurality of supporting ribs disposed within the chamber between the bottom chassis and the radiolucent top surface and configured to support the radiolucent top surface during displacement of the X-ray detector;
a coupling mechanism associated with the plurality of supporting ribs and configured to maintain spacing among the supporting ribs during displacement of the X-ray detector; and
a locking mechanism configured to secure the X-ray detector at a selected position within the chamber.

20. The integrated detector platform of claim 19, further comprising a control system operably connected to the displacement mechanism, the control system configured to receive a positioning input and to drive the displacement mechanism to position the X-ray detector based on the positioning in
